(19) United States
(12) Patent Application Publication
(10) Pub. No.: US 2022/0387286 A1
(43) Pub. Date: Dec. 8, 2022

(54) COMPOSITIONS AND METHODS FOR KERATIN FIBERS

(71) Applicant: L'OREAL, Paris (FR)

(72) Inventors: Rita CHOKSHI, Monroe Township, NJ (US); Seyma ASLAN, Clifton, NJ (US)

(21) Appl. No.: 17/332,627

(22) Filed: May 27, 2021

(51) Int. Cl. A61K 8/73; A61K 8/60; A61K 8/34; A61K 8/81; A61K 8/362; A61K 8/365; A61K 8/92; A61K 8/46; A61K 8/04; A61K 8/86; A61Q 5/06

(52) U.S. Cl. CPC A61K 8/737; A61K 8/731; A61K 8/732; A61K 8/604; A61K 8/342; A61K 8/345; A61K 8/8182; A61K 8/8158; A61K 8/362; A61K 8/365; A61K 8/922; A61K 8/466; A61K 8/042; A61K 8/046; A61K 8/86; A61Q 5/06; A61K 2800/524; A61K 2800/5422; A61K 2800/5426; A61K 2800/594; A61K 2800/87

(57) ABSTRACT

The disclosure relates to compositions for keratin fibers, which have a unique transformative nature that allows the composition to change from a gel to a foam when shear stress is applied to the gel. The disclosure also relates to packaging systems comprising the compositions, and methods of using the compositions.

### TECHNICAL FIELD

The present disclosure relates to compositions for caring for, styling, and/or shaping the hair, packaging systems comprising the compositions, and methods of using the compositions.

### BACKGROUND

Conventional hair styling compositions are typically in the forms of lotions, gels, mousses, creams, sprays, serums, wax, oils, clays, etc. It is understood that different forms of compositions can provide different benefits, yet can also present different drawbacks and/or challenges, such as, for example, when the particular composition is applied to hair.

For example, hair gels are a common form for styling compositions for curly hair because they are usually good at providing curl definition and fighting frizz. However, hair gels have a tendency to be thick or have heavy oils mixed in. Thick gels may be too heavy for fine hair and may weigh down the curls of curly hair. In addition, gels may be difficult to apply evenly throughout the hair, and may lead to a “crunchy” sensation on the hair.

Foam compositions, such as mousses, are another form of hair styling composition. There are two types of foam hair styling compositions, aerosol type and non-aerosol type. The aerosol type compositions typically require a propellant and the compositions are usually filled in an aerosol pressure resistant container and are dispensed from the nozzle of the container in the form of a foam. The non-aerosol type hair styling composition does not use a propellant, but the composition is typically filled in a foamer container and produces foam when shear stress is applied, e.g. through a pump. Currently existing non-aerosol hair styling compositions transform a liquid composition to a foam.

As such, there is a need for hair styling compositions that can be easily applied to hair, give good curl definition, provide long-lasting hold, hair manageability, and good cosmetic properties, and/or be environmentally friendly. Ideally, new galenic forms of compositions are desired.

It has now surprisingly been found that compositions according to the disclosure have a unique transformative nature wherein the composition is provided in the form of a gel that can transform into a foam under application of shear stress. These foam compositions can be easy to apply, provide good curl definition and/or hold, and/or may impart various advantageous sensory properties to the hair.

### BRIEF DESCRIPTION OF FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a graph illustrating rheological properties of a typical gel.

FIG. 2 is a graph illustrating viscosity properties of a transformative gel.

FIG. 3 is an image illustrating an exemplary texture transformation of a gel-to-foam composition under shear stress.

FIG. 4 is a graph showing how long various benefits lasted after hair was treated with a composition according to the disclosure.

It is to be understood that the foregoing and following descriptions are exemplary and explanatory only, and are not intended to be restrictive of any subject matter claimed.

### SUMMARY

The disclosure relates to compositions for caring for, styling, and/or shaping keratin fibers, e.g. human hair, which have a unique transformative nature such that the compositions can transform from a gel to a foam. Compositions disclosed herein can, in various embodiments, impart certain cosmetic properties to hair, such as one or more of styling/shaping, curl definition, frizz control, discipline, hydration, moisture, smoothness, shine, and/or conditioning. The disclosure also relates to packaging systems comprising the compositions, and methods of using the compositions.

In various embodiments, the disclosure relates to gel or foam compositions comprising (a) at least one nonionic guar polymer; (b) at least one polysaccharide other than the at least one nonionic guar polymer; (c) at least one nonionic film forming polymer; (d) at least one cationic film-forming polymer; (e) at least one anionic surfactant; and (f) at least one polyol. In various embodiments, the weight ratio of the at least one polysaccharide other than the nonionic guar polymer to the at least one nonionic guar polymer ranges from about 1:0.01 to about 1:1, for example about 1:0.1 to about 1:0.5, such as about 1:0.1, about 1:0.15, about 1:0.2, about 1:0.25, or about 1:0.3.

In various embodiments, the at least one nonionic guar polymer comprises a nonionic hydroxyalkyl-modified guar polymer, for example hydroxypropyl guar. In some embodiments, the at least one nonionic guar polymer is present in an amount ranging from about 0.01% to about 2%, such as about 0.1% to about 1%, or about 0.2% to about 0.4% by weight, relative to the total weight of the composition.

In further embodiments, the at least one polysaccharide other than the at least one nonionic guar polymer is chosen from gums, celluloses, starches, or mixtures thereof, for example xanthan gum. In some embodiments, the at least one polysaccharide other than the at least one nonionic guar polymer is present in an amount ranging from about 0.01% to about 5%, such as about 0.1% to about 4%, or about 0.6% to about 1.2% by weight, relative to the total weight of the composition.

In various embodiments, the at least one nonionic film-forming polymer is chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, copolymers of acrylonitrile and of a non-ionic monomer, or mixtures thereof, for example VP/methacrylamide/vinyl imidazole copolymer. In some embodiments, the at least one nonionic film-forming polymer is present in an amount ranging from about 0.01% to about 5%, such as about 0.1% to about 3%, or about 0.4% to about 1% by weight, relative to the total weight of the composition.

In further embodiments, the at least one cationic film-forming polymer is chosen from quaternized or non-quaternized vinylpyrrolidone/dialkylaminoalkyl acrylate or methacrylate copolymers, fatty-chain polymers containing a vinylpyrrolidone unit, dimethylaminoethyl methacrylate/vinylcaprolactam/vinylpyrrolidone terpolymers, quaternized vinylpyrrolidone/dimethylaminopropylmethacrylamide copolymers, cationic guar gum derivatives, quaternary copolymers of vinylpyrrolidone and of vinylimidazole, chitosans or salts thereof, cationic cellulose derivatives, or mixtures thereof, for example quaternized or non-quaternized vinylpyrrolidone/dialkylaminoalkyl acrylate or methacrylate copolymers, e.g. VP/dimethylaminoethyl methacrylate copolymer, fatty-chain polymers containing a vinylpyrrolidone unit, dimethylaminoethyl methacrylate/vinylcaprolactam/vinylpyrrolidone terpolymers, quaternized vinylpyrrolidone/dimethylaminopropylmethacrylamide copolymers, cationic guar gum derivatives, quaternary copolymers of vinylpyrrolidone and of vinylimidazole, chitosans or salts thereof, cationic cellulose derivatives, or mixtures thereof. In some embodiments, the at least one cationic film-forming polymer is present in an amount ranging from about 0.01% to about 5%, such as about 0.1% to about 3%, or about 0.2% to about 1% by weight, relative to the total weight of the composition.

In various embodiments, the at least one anionic surfactant is chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof, for example a C₁₀-C₂₄ olefin sulfonate such as sodium C₁₄₋₁₆ olefin sulfonate. In some embodiments, the at least one anionic surfactant is present in an amount ranging from about 0.01% to about 6%, such as about 0.1% to about 3%, or about 0.5% to about 1.5% by weight, relative to the total weight of the composition.

In various embodiments, the at least one polyol is chosen from one or more alkane polyols, for example glycerine, caprylyl glycol, or a mixture thereof. In some embodiments, the at least one polyol is present in an amount ranging from about 0.1% to about 10%, such as about 1% to about 7%, or about 3% to about 5% by weight, relative to the total weight of the composition.

Optionally, the compositions may further comprise at least one alkylpolyglucoside, such as, for example decylglucoside. If present, the alkylpolyglucoside may be present in an amount ranging from about 0.1% to about 8%, such as about 0.5% to about 5%, or about 0.75% to about 3% by weight, relative to the total weight of the composition.

The compositions may optionally further comprise additional components, such as, for example, one or more solvents (e.g. water and/or non-aqueous solvents), one or more conditioning agents (e.g. oils, such as castor oil, and/or plant extracts), one or more active agents (e.g. acids, such as citric acid and/or maleic acid), one or more vitamins (e.g. panthenol and/or tocopherol), and/or one or more preservatives.

In further embodiments, the disclosure relates to gel or foam compositions comprising (a) from about 0.01% to about 1% by weight of at least one nonionic hydroxyalkyl-modified guar polymer; (b) from about 0.01% to about 3% by weight of at least one polysaccharide other than the at least one nonionic guar polymer chosen from gums, celluloses, starches, or mixtures thereof; (c) from about 0.01% to about 2% by weight of at least one nonionic film-forming polymer; (d) from about 0.1% to about 4% by weight of at least one cationic film-forming polymer; (e) from about 0.01% to about 3% by weight of at least one anionic surfactant chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof; and (f) from about 2% to about 6% by weight at least one polyol chosen from alkane polyols. Optionally, the compositions may further comprise (g) from about 0.01% to about 1% of at least one vitamin; and (h) from about 0.5% to about 5% of at least one alkylglucoside. The compositions may further comprise at least one solvent and/or at least one conditioning agent. In various embodiments, the weight ratio of the at least one polysaccharide other than the nonionic guar polymer to the at least one nonionic guar polymer ranges from about 1:0.01 to about 1:1, for example about 1:0.1 to about 1:0.5, such as about 1:0.1, about 1:0.15, about 1:0.2, about 1:0.25, or about 1:0.3.

In still further embodiments, the disclosure relates to gel or foam compositions comprising (a) from about 0.2% to about 0.4% by weight of at least one nonionic hydroxyalkyl-modified guar polymer; (b) from about 0.6% to about 1.2% by weight of at least one polysaccharide other than the at least one nonionic guar polymer, chosen from gums, celluloses, starches, or mixtures thereof; (c) from about 0.4% to about 1% by weight of at least one nonionic film-forming polymer; (d) from about 0.2% to about 1% by weight of at least one cationic film-forming polymer; (e) from about 0.5% to about 1.5% by weight of at least one anionic surfactant chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof; (f) from about 1% to about 7% by weight at least one polyol chosen from alkane polyols; (g) from about 0.2% to about 0.5% of at least one vitamin; and (h) from about 0.75% to about 3% of at least one alkylglucoside. The compositions may further comprise at least one solvent and/or at least one conditioning agent. In various embodiments, the weight ratio of the at least one polysaccharide other than the nonionic guar polymer to the at least one nonionic guar polymer ranges from about 1:0.01 to about 1:1, for example about 1:0.1 to about 1:0.5, such as about 1:0.1, about 1:0.15, about 1:0.2, about 1:0.25, or about 1:0.3.

In still further embodiments, the disclosure relates to gel or foam compositions comprising (a) from about 0.2% to about 0.4% by weight of at least one nonionic hydroxyalkyl-modified guar polymer; (b) from about 0.6% to about 1.2% by weight of at least one polysaccharide other than the at least one nonionic guar polymer chosen from gums, celluloses, starches, or mixtures thereof; (c) from about 0.4% to about 1% by weight of at least one nonionic film-forming polymer chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, copolymers of acrylonitrile and of a non-ionic monomer, or mixtures thereof; (d) from about 0.2% to about 1% by weight of at least one cationic film-forming polymer chosen from quaternized or non-quaternized vinylpyrrolidone/dialkylaminoalkyl acrylate or methacrylate copolymers, fatty-chain polymers containing a vinylpyrrolidone unit, dimethylaminoethyl methacrylate/vinylcaprolactam/vinylpyrrolidone terpolymers, quaternized vinylpyrrolidone/dimethylaminopropylmethacrylamide copolymers, cationic guar gum derivatives, quaternary copolymers of vinylpyrrolidone and of vinylimidazole, chitosans or salts thereof, cationic cellulose derivatives, or mixtures thereof, for example quaternized or non-quaternized vinylpyrrolidone/dialkylaminoalkyl acrylate or methacrylate copolymers, e.g. VP/dimethylaminoethyl methacrylate copolymer, fatty-chain polymers containing a vinylpyrrolidone unit, dimethylaminoethyl methacrylate/vinylcaprolactam/vinylpyrrolidone terpolymers, quaternized vinylpyrrolidone/dimethylaminopropylmethacrylamide copolymers, cationic guar gum derivatives, quaternary copolymers of vinylpyrrolidone and of vinylimidazole, chitosans or salts thereof, cationic cellulose derivatives, or mixtures thereof; (e) from about 0.5% to about 1.5% by weight of at least one anionic surfactant chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof; (f) from about 1% to about 7% by weight at least one polyol chosen from alkane polyols; (g) optionally, from about 0.2% to about 0.5% of at least one vitamin; and (h) optionally, from about 0.75% to about 3% of at least one alkylglucoside. The compositions may further comprise at least one solvent and/or at least one conditioning agent. In various embodiments, the weight ratio of the at least one polysaccharide other than the nonionic guar polymer to the at least one nonionic guar polymer ranges from about 1:0.01 to about 1:1, for example about 1:0.1 to about 1:0.5, such as about 1:0.1, about 1:0.15, about 1:0.2, about 1:0.25, or about 1:0.3.

In further embodiments, the disclosure relates to packaging systems comprising a dispensing container containing a gel composition according to the disclosure. The packaging systems and/or dispensing containers may be configured to apply shear stress to the gel, in order to transform the gel into a foam.

In further embodiments, the disclosure relates to methods of caring for, styling, and/or shaping hair, the methods comprising (i) applying shear stress to a gel composition according to the disclosure, wherein after the shear stress is applied to the gel composition, the composition is in the form of a foam; and (ii) applying the foam to the hair. In some embodiments, the methods include a step of heating the hair before, during, or after application of the foam composition to the hair, although methods according to other embodiments do not include or require the use of heat before, during, or after application of the foam composition to the hair.

### DETAILED DESCRIPTION

The disclosure relates to compositions for caring for, styling, and/or shaping hair, packaging systems comprising the compositions, and methods of using the compositions.

The hair styling compositions according to the disclosure have unique textures of gels that can transform into foams upon application of shear stress. Typical gel compositions are better in consistency and stronger at hold, while typical foam compositions are easier to spread and have better coverage. Thus, compositions according to the disclosure provide benefits of both a gel and a foam.

I. Compositions

In exemplary and non-limiting embodiments, compositions according to the disclosure comprise (a) at least one nonionic guar polymer; (b) at least one polysaccharide other than the at least one nonionic guar polymer; (c) at least one nonionic film-forming polymer; (d) at least one cationic film-forming polymer; (e) at least one anionic surfactant; and (f) at least one polyol.

A propellant is not required for the compositions. As such, the compositions may include a propellant, or may be free or essentially free of propellant. The compositions may be further free or essentially free of silicones and/or sulfate-based anionic surfactants.

### Nonionic Guar Polymer

Compositions according to the disclosure comprise at least one nonionic guar polymer. Non-limiting examples of nonionic guar polymers that can be used include unmodified and modified guar polymers. For example, modified guar polymers include those that are modified by hydroxyalkyl groups, for example C1-C6 hydroxyalkyl groups. Non-limiting examples of hydroxyalkyl groups include hydroxymethyl, hydroxyethyl, hydroxypropyl, and hydroxybutyl groups. For example, in one exemplary embodiment, the at least one nonionic guar polymer comprises, consists essentially of, or consists of hydroxypropyl guar.

Non-limiting examples of nonionic, unmodified guar gums that may be used in various embodiments include Guargel D/15 (Noveon); Vidogum GH 175 (Unipectine), Meypro-Guar 50 and JAGUAR® C (Meyhall/Rhodia Chimie). Non-limiting examples of nonionic modified guar gums include Jaguar® HP8, HP60, HP120, DC 293 and HP 105 (Meyhall/Rhodia Chimie); Galactasol 4H4FD2 (Ashland); and N-Hance® HP by the company Aqualon.

In various exemplary embodiments, the total amount of the at least one nonionic guar polymer may vary, but typically ranges from about 0.01% to about 2%, including all subranges therebetween, such as from about 0.01% to about 1.9%, from about 0.01% to about 1.8%, from about 0.01% to about 1.7%, from about 0.01% to about 1.6%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, from about 0.01% to about 0.1%, from about 0.1% to about 2%, from about 0.1% to about 1.9%, from about 0.1% to about 1.8%, from about 0.1% to about 1.7%, from about 0.1% to about 1.6%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, from about 0.1% to about 0.2%, from about 0.2% to about 2%, from about 0.2% to about 1.9%, from about 0.2% to about 1.8%, from about 0.2% to about 1.7%, from about 0.2% to about 1.6%, from about 0.2% to about 1.5%, from about 0.2% to about 1.4%, from about 0.2% to about 1.3%, from about 0.2% to about 1.2%, from about 0.2% to about 1%, from about 0.2% to about 0.9%, from about 0.2% to about 0.8%, from about 0.2% to about 0.7%, from about 0.2% to about 0.6%, from about 0.2% to about 0.5%, from about 0.2% to about 0.4%, or from about 0.2% to about 0.3% by weight, relative to the total weight of the composition, including all ranges and subranges thereof.

### Polysaccharide

The compositions further comprise at least one polysaccharide other than the at least one nonionic guar polymer. Polysaccharides are polymers which exhibit monosaccharides or disaccharides as base units. The polysaccharides other than the at least one nonionic guar polymer which can be used in the compositions according to the present invention include, by way of example only, gums, celluloses, and starches.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxyethylcelluloses, hydroxypropylmethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose.

Non-limiting examples of starches include modified starches, starch-based polymers, methylhydroxypropyl starch, potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In various exemplary embodiments, the total amount of the one or more polysaccharide other than the nonionic guar polymer may vary, but is typically ranges from about 0.01% to about 5%, including all subranges therebetween, such as from about 0.01% to about 4%, from about 0.01% to about 3%, from about 0.01% to about 2%, from about 0.01% to about 1.8%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.1% to about 5%, from about 0.1% to about 4%, from about 0.1% to about 3%, from about 0.1% to about 2%, from about 0.1% to about 1.8%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.2% to about 5%, from about 0.2% to about 4%, from about 0.2% to about 3%, from about 0.2% to about 2%, from about 0.2% to about 1.8%, from about 0.2% to about 1.5%, from about 0.2% to about 1.4%, from about 0.2% to about 1.3%, from about 0.2% to about 1.2%, from about 0.2% to about 1.1%, from about 0.2% to about 1%, from about 0.4% to about 5%, from about 0.4% to about 4%, from about 0.4% to about 3%, from about 0.4% to about 2%, from about 0.4% to about 1.8%, from about 0.4% to about 1.5%, from about 0.4% to about 1.4%, from about 0.4% to about 1.3%, from about 0.4% to about 1.2%, from about 0.4% to about 1.1%, from about 0.4% to about 1%, from about 0.6% to about 5%, from about 0.6% to about 4%, from about 0.6% to about 3%, from about 0.6% to about 2%, from about 0.6% to about 1.8%, from about 0.6% to about 1.5%, from about 0.6% to about 1.4%, from about 0.6% to about 1.3%, from about 0.6% to about 1.2%, from about 0.6% to about 1.1%, or from about 0.6% to about 1%, by weight, relative to the total weight of the composition, including all ranges and subranges thereof. In at least certain embodiments, the composition comprises a polysaccharide other than the nonionic guar polymer in an amount less than about 2%, such as less than about 1.75%, less than about 1.5%, less than about 1.25%, or less than about 1% by weight, relative to the total weight of the composition. In other embodiments, the at least one polysaccharide other than the nonionic guar polymer in compositions according to the disclosure is present in an amount ranging from about 0.5% to about 1.5%, or about 0.6% to about 1.2% by weight, relative to the total weight of the composition. In at least one embodiment, the at least one polysaccharide other than the nonionic guar polymer comprises, consists essentially of, or consists of xanthan gum.

In various exemplary embodiments, the weight ratio of the at least one polysaccharide other than the nonionic guar polymer to the at least one nonionic guar polymer ranges from about 1:0.01 to about 1:1, such as for example ranging from 1:0.05 to about 1:1, from about 1:0.1 to about 1:0.8, or from about 1:0.1 to about 1:0.5, including all ranges and subranges thereof. In at least one exemplary embodiment, the weight ratio of at least one polysaccharide other than the nonionic guar polymer to the at least one nonionic guar polymer is about 1:0.1, about 1:0.15, about 1:0.2, about 1:0.25, or about 1:0.3.

### Nonionic and Cationic Film-Forming Polymers

Compositions according to the present disclosure further comprise at least one nonionic film-forming polymer and at least one cationic film-forming polymer. In combination, these polymers may provide improved curl definition and/or styling properties.

Non-limiting examples of useful nonionic film-forming polymers include polyalkyloxazolines, such as the polyethyloxazolines provided by the company Polymer Chemistry Innovations under the names Aquazol HP, and Aquzol HVIS; vinyl acetate homopolymers, such as the product provided under the name UCAR 130 Latex Resin by the company Dow Chemical or the product provided under the name Ultrapure Polymer 2041-R 012 by the company Ultra Chemical, Inc.; vinyl acetate copolymers, for instance copolymers of vinyl acetate and of acrylic ester, such as the product provided under the name Rhodopas AD 310 from Rhone-Poulenc; copolymers of vinyl acetate and of ethylene, such as the product provided under the name Dermacryl LOR by the company Akzo Nobel, or copolymers of vinyl acetate and of maleic ester, for example of dibutyl maleate, such as the product provided under the name Appretan MB Extra by the company Clariant; alkyl acrylate homopolymers and alkyl methacrylate homopolymers, such as the product provided under the name Micropearl RQ 750 by the company Matsumoto or the product provided under the name Luhydran® A 848 S by the company BASF; copolymers of polyethylene and of maleic anhydride; homopolymers and copolymers of acrylic esters, for instance copolymers of alkyl acrylates and of alkyl methacrylates, such as the products provided by the company Rohm GmbH under the name Eudragit® NE 30 D (INCI name Acrylates copolymer); copolymers of acrylonitrile and of a non-ionic monomer, chosen, for example, from butadiene and alkyl (meth)acrylates; styrene homopolymers; styrene copolymers, for instance copolymers of styrene, of alkyl acrylate and of alkyl methacrylate; copolymers of styrene and of butadiene, or copolymers of styrene, of butadiene and of vinylpyridine; polyamides; vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF, polyvinylpyrrolidone (INCI name PVP); vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); polyurethanes, such as the products provided under the names Acrysol RM 1020 or Acrysol RM 2020 by the company Dow Chemical or the products Uraflex XP 401 UZ or Uraflex XP 402 UZ by the company DSM Resins; copolymers of alkyl acrylate and of urethane, such as the product 8538-33 by the company National Starch; and polyamides, such as the product Estapor LO 11 provided by the company Rhone-Poulenc; and vinylpyrrolidone/methacrylamide/vinylimidazole terpolymers, for instance the product sold under the name Luviset® Clear by the company BASF (INCI name VP/methacrylamide/vinyl imidazole copolymer).

In various exemplary embodiments, the amount of the nonionic film-forming polymer(s) may vary, but typically ranges from about 0.01% to about 5%, including all subranges therebetween, such as from about 0.01% to about 4%, from about 0.01% to about 3%, from about 0.01% to about 2.5%, from about 0.01% to about 2%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.01% to about 0.9%, from about 0.01% to about 0.8%, from about 0.1% to about 5%, from about 0.1% to about 4%, from about 0.1% to about 3%, from about 0.1% to about 2.5%, from about 0.1% to about 2%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.1% to about 0.9%, from about 0.1% to about 0.8%, from about 0.4% to about 5%, from about 0.4% to about 4%, from about 0.4% to about 3%, from about 0.4% to about 2.5%, from about 0.4% to about 2%, from about 0.4% to about 1.5%, from about 0.4% to about 1.4%, from about 0.4% to about 1.3%, from about 0.4% to about 1.2%, from about 0.4% to about 1.1%, from about 0.4% to about 1%, from about 0.4% to about 0.9%, or from about 0.4% to about 0.8% by weight, relative to the total weight of the composition, including all ranges and subranges thereof. In at least certain embodiments, the at least one nonionic polymer is present in an amount ranging from about 0.2% to about 2% by weight, such as about 0.2% to about 1%, about 0.2% to about 0.8%, about 0.5% to about 2%, about 0.5% to about 1%, or about 0.5% to about 8% by weight, relative to the total weight of the composition, including all ranges and subranges thereof.

By way of non-limiting example, the cationic film-forming polymers that can be used in the may be chosen from polymers comprising primary, secondary, tertiary and/or quaternary amine groups forming part of the polymer chain or directly attached thereto, and having a molecular weight of ranging from 500 and about 5,000,000 and preferably ranging from 1000 and 3,000,000. Among these polymers, mention may be made more particularly of the following cationic film-forming polymers:

(1) homopolymers or copolymers derived from acrylic or methacrylic esters or amides and comprising at least one of the following units (A)-(D):

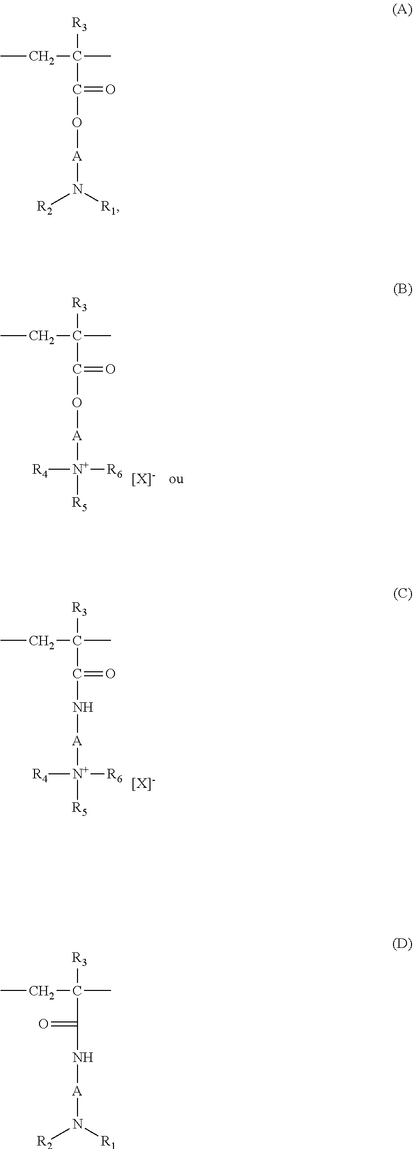

in which:
R₃ denotes a hydrogen atom or a CH₃ radical;
A is a linear or branched alkyl group comprising from 1 to 6 carbon atoms or a hydroxyalkyl group comprising from 1 to 4 carbon atoms;
R₄, R₅ and R₆, which may be identical or different, represent an alkyl group having from 1 to 18 carbon atoms or a benzyl radical;
R₁ and R₂, which may be identical or different, each represent a hydrogen atom or an alkyl group having from 1 to 6 carbon atoms; and
X denotes a methosulfate anion or a halide such as chloride or bromide.

The copolymers of family (1) also contain one or more units derived from comonomers that may be chosen from the family of acrylamides, methacrylamides, diacetone acrylamides, acrylamides and methacrylamides substituted on the nitrogen with lower (C₁-C₄) alkyl groups, groups derived from acrylic or methacrylic acids or esters thereof, vinyllactams such as vinylpyrrolidone or vinylcaprolactam, and vinyl esters. Thus, among these copolymers of family (1), mention may be made of:
quaternized or non-quaternized vinylpyrrolidone/dialkylaminoalkyl acrylate or methacrylate copolymers, such as the products sold under the name Gafquat® by the company ISP, for instance Gafquat® 734 or Gafquat® 755 or Gafquat® 755N (INCI name Polyquaternium-11), or alternatively the products known as Copolymer® 845, 958 and 937 sold by ISP (INCI name VP/dimethylaminoethyl methacrylate copolymer). These polymers are described in detail in French patents 2077143 and 2393573;
fatty-chain polymers containing a vinylpyrrolidone unit, such as the products sold under the name Styleze® W20L and Styleze® W10 by the company ISP (INCI name Polyquaternium-55);
dimethylaminoethyl methacrylate/vinylcaprolactam/vinylpyrrolidone terpolymers, such as the products sold under the names Advantage HC 37 or Gaffix® VC 713 by the company ISP (INCI name Vinyl caprolactam/VP/dimethylaminoethyl methacrylate copolymer); and
quaternized vinylpyrrolidone/dimethylaminopropylmethacrylamide copolymers, such as the products sold under the name Gafquat® HS 100 by the company ISP (name Polyquaternium-28);

(2) cationic guar gum derivatives, preferably containing quaternary ammonium, such as those described in U.S. Pat. Nos. 3,589,578 and 4,031,307, such as guar gums containing trialkylammonium cationic groups. Such products are sold in particular under the trade names Jaguar® C13 S, Jaguar® C 15 and Jaguar® C 17 by the company Rhodia (INCI name Guar hydroxypropyltrimonium chloride);

(3) quaternary copolymers of vinylpyrrolidone and of vinylimidazole; mention may be made, for example, of vinylpyrrolidone/methylvinylimidazolium chloride copolymers, such as the products sold by the company BASF under the names Luviquat® FC550 or FC370, Luviquat® Excellence and Luviquat® Style (INCI name Polyquaternium-16), or vinylpyrrolidone/vinylimidazolium methosulfate/vinylcaprolactam terpolymers, such as the product Luviquat® Hold sold by the company BASF (INCI name Polyquaternium-46);

(4) chitosans or salts thereof; the salts that can be used are, in particular, chitosan acetate, lactate, glutamate, gluconate or pyrrolidonecarboxylate. Among these compounds, mention may be made of the chitosan pyrrolidonecarboxylate sold under the name Kytamer® PC by the company Amerchol (INCI name Chitosan PCA); and

(5) cationic cellulose derivatives such as copolymers of cellulose or of cellulose derivatives grafted with a water-soluble monomer comprising a quaternary ammonium, and described in particular in U.S. Pat. No. 4,131,576, such as hydroxyalkylcelluloses, for instance hydroxymethyl-, hydroxyethyl- or hydroxypropylcelluloses grafted in particular with a methacryloyloxyethyltrimethylammonium, methacrylamidopropyltrimethyl-ammonium or dimethyldiallylammonium salt. The commercial products corresponding to this definition are, more particularly, the products sold under the name Celquat® L 200 and Celquat® H 100 by the company Akzo Nobel (INCI name Polyquaternium-4).

The amount of the cationic film-forming polymer(s) may vary, but typically ranges from about 0.01% to about 5%, including all subranges therebetween, such as from about 0.01% to about 4%, from about 0.01% to about 3%, from about 0.01% to about 2.5%, from about 0.01% to about 2%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.01% to about 0.9%, from about 0.01% to about 0.8%, from about 0.05% to about 5%, from about 0.05% to about 4%, from about 0.05% to about 3%, from about 0.05% to about 2.5%, from about 0.05% to about 2%, from about 0.05% to about 1.5%, from about 0.05% to about 1.4%, from about 0.05% to about 1.3%, from about 0.05% to about 1.2%, from about 0.05% to about 1.1%, from about 0.05% to about 1%, from about 0.05% to about 0.9%, from about 0.05% to about 0.8%, from about 0.1% to about 5%, from about 0.1% to about 4%, from about 0.1% to about 3%, from about 0.1% to about 2.5%, from about 0.1% to about 2%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.1% to about 0.9%, from about 0.1% to about 0.8%, from about 0.2% to about 5%, from about 0.2% to about 4%, from about 0.2% to about 3%, from about 0.2% to about 2.5%, from about 0.2% to about 2%, from about 0.2% to about 1.5%, from about 0.2% to about 1.4%, from about 0.2% to about 1.3%, from about 0.2% to about 1.2%, from about 0.2% to about 1.1%, from about 0.2% to about 1%, from about 0.2% to about 0.9%, or from about 0.2% to about 0.8% by weight, relative to the total weight of the composition, including all ranges and subranges thereof. In at least certain embodiments, the at least one nonionic polymer is present in an amount ranging from about 0.05% to about 1.8% by weight, such as about 0.1% to about 0.7%, or about 0.3% to about 0.7% by weight, relative to the total weight of the composition, including all ranges and subranges thereof.

### Anionic Surfactants

Compositions according to the disclosure comprise at least one anionic surfactant. In various embodiments, anionic surfactants included in the compositions disclosed herein are non-sulfate anionic surfactants.

Useful anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples are provided below.

### Acyl Isethionates

Non-limiting examples of useful acyl isethionates and their salts include those of formula (Ia) and (Ib):

RCOOCHR¹CHR²X⁻M⁺ (Ia)

RCOOCHR¹CHR²X⁻Na⁺ (Ib)

wherein:
R, R¹, and R² are each independently chosen from H or an alkyl chain having 1-24 carbon atoms, said chain being saturated or unsaturated, linear or branched;
X is COO⁻ or SO₃⁻; and
M is any suitable cation.

Although the cation may be chosen from any suitable cation including, for example, alkali metal ion such as sodium or potassium, ammonium ions, or alkanolammonium ions such as monoethanolammonium or triethanolammonium ions, sodium is a preferred cation. In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, sulfonates of alkylphenolpolyglycol ethers, alkylbenzenesulfonates, phenylalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (II) is particularly useful:

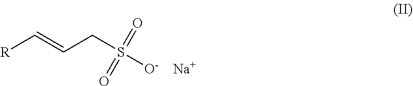

wherein R is selected from H or alkyl chain that has 1-24 carbon atoms, preferably 6-24 carbon atoms, more preferably, 8 to 20 carbon atoms, said chain being saturated or unsaturated, linear or branched, substituted or unsubstituted. Sodium is shown as the cation in the above formula (II) but the cation may be may be chosen from any suitable cation including, for example, alkali metal ion such as sodium or potassium, ammonium ions, or alkanolammonium ions such as monoethanolammonium or triethanolammonium ions.

In some instances, the alkyl sulfonate(s) are selected from C8-C16 alkyl benzene sulfonates, C10-C20 paraffin sulfonates, C₁₀-C₂₄ olefin sulfonates, salts thereof, or mixtures thereof. In certain embodiments, C₁₀-C₂₄ olefin sulfonates and salts thereof may be preferred. A non-limiting example of a C₁₀-C₂₄ olefin sulfonate that can be used is sodium C₁₄₋₁₆ olefin sulfonate.

### Alkyl Sulfosuccinates

Non-limiting examples of useful alkyl sulfosuccinates and their salts include those of formula (III):

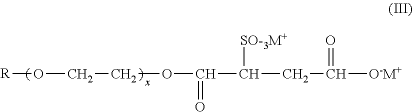

wherein:
R is a straight or branched chain alkyl or alkenyl group having 10 to 22 carbon atoms, preferably 10 to 20 carbon atoms;
x is a number that represents the average degree of ethoxylation, and can range from 0 to about 5, preferably from 0 to about 4, and most preferably from about 2 to about 3.5; and
M, which can be the same or different, is chosen from any suitable monovalent cation.

In some embodiments, cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

### Alkyl Sulfoacetates

Non-limiting examples of alkyl sulfoacetates and their salts include, for example, alkyl sulfoacetates such as C4-C18 fatty alcohol sulfoacetates and/or salts thereof. In some embodiments, a sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (IV):

R—O[CH₂O]ᵤ[(CH₂)ₓCH(R′)(CH₂)_{y}(CH₂)_{z}O]ᵥ[CH₂CH₂O]_{w}CH₂COOH (IV)

wherein:
R is a hydrocarbon radical containing from about 6 to about 40 carbon atoms; R′ represents hydrogen or alkyl;
u, v, and w, which may be identical or different, independently represent numbers from 0 to 60;
x, y, and z, which may be identical or different, independently represent numbers from 0 to 13; and
the sum of x+y+z>0.

Compounds corresponding to formula (IV) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (IV), R is linear or branched, acyclic or cyclic, saturated or unsaturated, aliphatic or aromatic, substituted or unsubstituted. For example, R may be a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, may be chosen from a number ranging from 2 to 20, such as a number ranging from 3 to 17, or a number ranging from 5 to 15. Further still, x, y, z, independently of one another, may be chosen from a number ranging from 0 to 13, such as a number ranging from 1 to 10, or a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof. In some cases, preferred ethoxylated acids include Oleth-10 Carboxylic Acid, Laureth-5 Carboxylic Acid, Laureth-11 Carboxylic Acid, or mixtures thereof.

### Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (V):

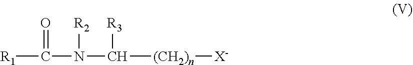

wherein:
R¹, R², and R³ are each independently selected from H or an alkyl chain having 1-24 carbon atoms, said chain being saturated or unsaturated, linear or branched, substituted or unsubstituted;
n ranges from 0 to 30; and
X is COO⁻ or SO₃⁻.

### Acyl Taurates

Non-limiting examples of acyl taurates include those of formula (VI):

RCO—NR¹CHR²CHR³SO₃Na (VI)

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (VII):

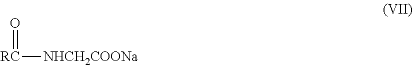

wherein R is an alkyl chain of 8 to 16 carbon atoms. Sodium is shown as the cation in the above formula (VII), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate.

### Acyl Glutamates

Non-limiting examples of useful acyl glutamates include those of formula (VIII):

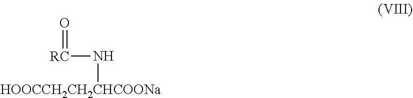

wherein R is an alkyl chain of 8 to 16 carbon atoms. Sodium is shown as the cation in the above formula (VIII) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In various embodiments, the total amount of the at least one anionic surfactant in a composition disclosed herein may vary, but typically ranges from about 0.01% to about 6% by weight, including all subranges therebetween, such as from about 0.01% to about 5%, from 0.01% to about 4%, from about 0.01% to about 3%, from about 0.01% to about 2%, from about 0.01% to about 1.5%, from about 0.01% to about 1%, from about 0.1% to about 6%, from about 0.1% to about 5%, from 0.1% to about 4%, from about 0.1% to about 3%, from about 0.1% to about 2%, from about 0.1% to about 1.5%, from about 0.1% to about 1%, from about 0.5% to about 6%, from about 0.5% to about 5%, from about 0.5% to about 4%, from about 0.5% to about 3%, from about 0.5% to about 2%, from about 0.5% to about 1.5%, from about 0.5% to about 1%, or from about 0.75% to about 1% by weight, relative to the total weight of the composition, including all ranges and subranges thereof. In some particular embodiments, the total amount of the at least one anionic surfactant in a composition disclosed herein may range from about 0.5% to about 1.5%, such as about 0.8% to about 1.25% by weight, relative to the total weight of the composition, including all ranges and subranges thereof.

### Polyols

Compositions according to the disclosure comprise at least one polyol. In certain embodiments, compositions comprise at least two polyols. The term “polyol,” as used herein, refers to an organic molecule comprising at least two free hydroxy groups, such as, for example, at least three free hydoxy groups. The polyols may be liquid at ambient temperature (25° C.). The polyols may have from 2 to 32 carbon atoms, such as from 3 to 16 carbon atoms, or from 3 to 12 carbon atoms.

By way of example, the at least one polyol may be chosen from sugar alcohols or alkane polyols. Mixtures of polyols, such as mixtures of sugar alcohols and/or alkane polyols, may also be used. In certain embodiments, compositions comprise at least two polyols wherein at least one polyol is chosen from sugar alcohols, and at least one polyol is chosen from alkane polyols.

Sugar alcohols that can be used include but are not limited to sorbitol, maltitol, maltotriitol, erythritol, arabitol, adonitol, dulcitol, glucose, fructose, xylose, trehalose, sucrose, maltose, saccharose, lactose, and other hydrogenated oligosaccharides and polysaccharides, and mixtures thereof.

Exemplary and non-limiting alkane polyols that can be used in the compositions according to the present disclosure include glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol, or a mixture thereof. In a preferred embodiment, the compositions comprise glycerin, caprylyl glycol, or a mixture thereof.

The amount of the at least one polyol may vary but typically ranges from about 0.01% to about 15%, including all subranges therebetween, such as from about 0.1% to about 15%, from about 0.1% to about 12%, from about 0.1% to about 10%, from about 0.1% to about 8%, from about 0.1% to about 5%, from about 0.1% to about 4%, from about 0.1% to about 3%, from about 0.1% to about 2%, from about 0.1% to about 1.5%, from about 0.1% to about 1%, from about 0.5% to about 15%, from about 0.5% to about 12%, from about 0.5% to about 10%, from about 0.5% to about 8%, from about 0.5% to about 5%, from about 0.5% to about 4%, from about 0.5% to about 3%, from about 0.5% to about 2%, from about 0.5% to about 1.5%, from about 0.5% to about 1%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, or from about 1.5% to about 2% by weight, relative to the total weight of the composition, including all ranges and subranges thereof. For example, the at least one polyol may be present in an amount ranging from about 1% to about 9%, such as about 1.5% to about 8.5%, or from about 2% to about 6% by weight, relative to the total weight of the composition.

In certain exemplary embodiments, the at least one polyol is an alkane polyol, and is present in the composition in an amount ranging from about 0.1% to about 15%, such as from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, or from about 1% to about 2% by weight, relative to the total weight of the composition, including all ranges and subranges thereof.

In some embodiments, compositions according to the disclosure comprise one or more alkane polyol(s) present in the composition in an amount ranging from about 0.1% to about 10%, such as from about 1% to about 7%, about 2% to about 6%, or about 3% to about 5% by weight, relative to the total weight of the composition, including all ranges and subranges thereof, and the alkane polyol(s) comprises, consists essentially of, or consists of glycerin, caprylyl glycol, or a mixture thereof.

### Additional Components

Compositions according to the disclosure may optionally comprise additional components, including but not limited to solvents, conditioning agents, glucosides/polyglucosides, active agents, preservatives, and auxiliary components.

### Solvents

Compositions according to the disclosure optionally comprise a solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises one or more non-aqueous solvents, other than or in addition to polyols discussed above. For example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkane polyols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

The solvent may be present in the composition in an amount ranging from about 50% to about 99% by weight, relative to the total weight of the composition, including all ranges and subranges therebetween. For example, in one embodiment, the total amount of solvent may be about 65% to about 95%, about 65% to about 90%, about 70% to about 95%, about 70% to about 90%, about 75% to 95%, or about 75% to 90% by weight, relative to the total weight of the composition. In certain embodiments, the solvent is primarily comprised of water, such as from about 90% to about 99.99%, such as from about 95% to about 99.9%, or about 98% to about 99.9%, of the total solvent, with the remainder being comprised of non-aqueous solvents.

### Glucosides/Polyglucosides

Compositions according to the disclosure optionally comprise at least one alkyl or polyalkyl glucoside or polyglucoside.

Non-limiting alkyl and polyalkyl glucosides or polyglucosides include those containing an alkyl group comprising from 6 to 30 carbon atoms and preferably from 6 to 18 or even from 8 to 16 carbon atoms, and containing a glucoside group preferably comprising from 1 to 5 and especially 1, 2, or 3 glucoside units.

In certain embodiments, exemplary and useful alkyl polyglucosides include those having the following formula (IX):

R¹—O—(R²O)ₙ—Z(x) (IX)

wherein:
R¹ is an alkyl group having 8-18 carbon atoms;
R² is an ethylene or propylene group;
Z is a saccharide group with 5-6 carbon atoms;
n is an integer ranging from 0 to 10; and
x is an integer ranging from 1 to 5.

The alkylpolyglucosides may be chosen, for example, from decylglucoside, for instance the product sold under the name MYDOL10® by the company Kao Chemicals or the product sold under the name PLANTACARE® 2000 UP by the company BASF and the product sold under the name ORAMIX™ NS 10 by the company SEPPIC; caprylyl/capryl glucoside, for instance the product sold under the name PLANTACARE® KE 3711 by the company Cognis or ORAMIX™ CG 110 by the company SEPPIC; laurylglucoside, for instance the product sold under the name PLANTACARE® 1200 UP by the company BASF or PLANTAREN 1200 N® by the company BASF; cocoglucoside, for instance the product sold under the name PLANTACARE® 818 UP by the company BASF; caprylylglucoside, for instance the product sold under the name PLANTACARE® 810 UP by the company BASF, octyl glucoside, or mixtures thereof.

If present, the alkyl or polyalkyl glucoside(s) or polyglucoside(s) may be present in an amount ranging from about 0.1% to about 8%, such as about 0.5% to about 5%, about 0.75% to about 3%, or about 1% to about 2.5% by weight, relative to the total weight of the composition.

### Active Agents

In various embodiments, compositions according to the disclosure optionally comprise at least one active agent such as an acid, to provide optimized strengthening benefits to the hair. Non-limiting examples of useful acids include glycolic acid, lactic acid, maleic acid, malic acid, tartaric acid, citric acid, ascorbic acid, mandelic acid, azelaic acid, glyceric acid, tartronic acid, gluconic acid, benzylic acid, pyruvic acid, 2-hydroxybutyric acid, salicylic acid, trichloroacetic acid, or mixtures thereof.

The acids are typically non-polymeric and may have one (mono), two (di), or three (tri) carboxylic acid groups (—COOH). The non-polymeric mono-, di-, and tricarboxylic acids, and/or salts thereof, typically have a molecular weight of less than about 900 g/mol, such as less than about 700 g/mol, less than about 500 g/mol, less than about 300 g/mol, or less than about 200 g/mol.

Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof.

Non-limiting examples of tricarboxylic acids include citric acid, isocitric acid, aconitric acid, propane-1,2,3-tricarboxylic acid, benzene-1,3,5-tricarboxylic acid, a salt thereof, and a mixture thereof.

If present, the total amount of the one or more active agents may vary but typically ranges from about 0.0001% to about 10%, such as from about 0.0001% to about 5%, about 0.0001% to about 1%, about 0.001% to about 10%, about 0.001% to about 5%, about 0.001% to about 1% by weight, about 0.01% to about 10%, about 0.01% to about 5%, about 0.01% to about 1%, about 0.1% to about 10%, about 0.1% to about 5%, or about 0.1% to about 1% by weight, based on the total weight of the composition. For example, the total amount of the one or more acids may range from about 0.01% to about 0.5% by weight, based on the total weight of the composition.

### Conditioning Agents

Compositions according to the disclosure optionally comprise at least one conditioning agent other than silicone copolymers described above.

In various exemplary embodiments, the at least one conditioning agent may be chosen from non-silicone fatty compounds. The term “non-silicone fatty compound” means a fatty compound that does not contain any silicon atoms (Si). Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids, propylene glycol esters of fatty acids, butylene glycol esters of fatty acids, esters of neopentyl glycol and fatty acids, polyglycerol/glycerol esters of fatty acids, glycol diesters or diesters of ethylene glycol, fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of the fatty alcohols, fatty acids, fatty alcohol derivatives, and fatty acid derivatives are found in International Cosmetic Ingredient Dictionary, Sixteenth Edition, 2016, which is incorporated by reference herein in its entirety.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols.

In various exemplary embodiments, the conditioning agents may be chosen from cationic polymers, although in certain embodiments the compositions are free or essentially free of cationic polymers. The term “cationic polymer” means any polymer comprising at least one cationic group and/or at least one group that may be ionized into a cationic group.

In various embodiments, the total amount of at least one conditioning agent in the compositions may vary, but is typically from about 0.01% to about 10% by weight, including all subranges therebetween, such as from about 0.01% to about 8%, from about 0.01% to about 5%, from about 0.01% to about 3%, from about 0.01% to about 1%, from about 0.5% to about 10%, from about 0.5% to about 8%, from about 0.5% to about 5%, from about 1% to about 10%, from about 1% to about 8%, from about 1% to about 5% by weight, relative to the total weight of the composition. In some embodiments, the total amount of conditioning agents are present in an amount ranging from about 0.5% to about 2% by weight, based on the total weight of the composition.

### Preservatives

One or more preservatives may optionally be included in the compositions described herein. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), and a mixture thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, and a mixture thereof.

The total amount of the one or more preservatives, when present, may vary. In some cases, the total amount of the one or more preservatives is about 0.01% to about 5%, about 0.01% to about 4%, about 0.15% to about 1%, or about 1% to about 3%, by weight, relative to the total weight of the composition.

### Auxiliary Components

Compositions according to the disclosure optionally comprise any auxiliary component suitable for use in such compositions. Such components may include, but are not limited to, dyes/pigments, moisturizing agents, fatty substances, thickeners other than those previously described, fillers, structuring agents, shine agents, antioxidants or reducing agents, penetrants, sequestrants, fragrances, buffers, dispersants, plant extracts (e.g. fruit extracts, for example Pyrus Malus (Apple) Fruit Extract, and Aloe Barbadensis Leaf Juice Powder), opacifiers, sunscreen agents, vitamins (e.g. panthenol, tocopherol), pH adjusting agents, and antistatic agents.

Optional auxiliary components may be present in an amount ranging up to about 15% by weight, such as from about 0.01% to about 10%, or about 0.1% to about 5% by weight, relative to the total weight of the composition.

In some embodiments, the compositions comprise panthenol. In various embodiments, the panthenol may be present in an amount ranging from about 0.01% to about 1%, such as about 0.01% to about 0.5%, about 0.05% to about 0.75%, about 0.1% to about 0.75%, or about 0.2% to about 0.5%. In further embodiments, the compositions comprise tocopherol. Exemplary combined amounts of panthenol and tocopherol may range from about 0.01% to about 1%, such as about 0.01% to about 0.5%, about 0.05% to about 0.75%, about 0.1% to about 0.75%, or about 0.1% to about 0.5%.

The pH of the gel compositions is typically about 7.0 or lower, for example from about 2 to about 7, about 3 to about 6.5, about 3.5 to about 6, about 4 to about 6, about 4 to about 5.5, about 4.5 to about 5.5, or about 4.5 to about 5.

Optionally, the compositions may comprise up to about 100% biodegradable, sustainable, and/or environmentally friendly raw materials, such as up to about 99%, up to about 98%, up to about 97%, up to about 96%, or up to about 95%.

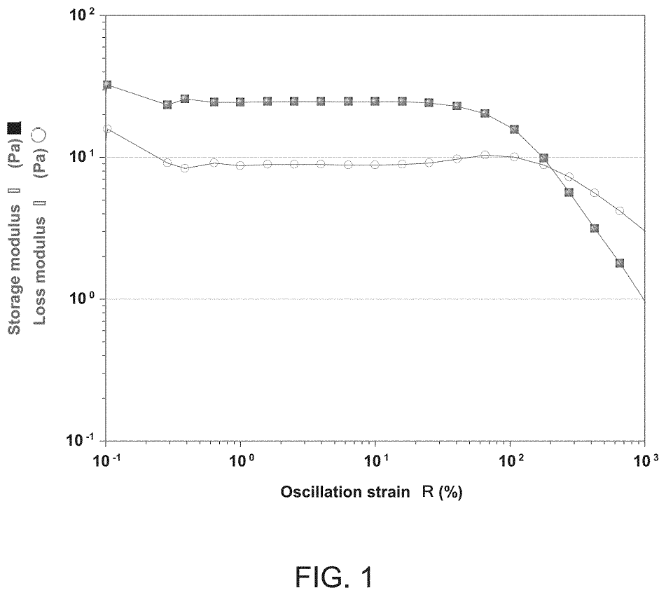

Compositions according to the disclosure are typically in the form of a gel prior to application of shear stress. The term “gel” is generally understood to mean a soft, semi-solid, or solid-like material that exhibits low to no flow under steady state. A gel can also be understood based on dynamic strain sweep test using a rheometer. FIG. 1 illustrates the typical rheological property of a general gel at the temperature where the composition would be applied to the hair. As shown in FIG. 1, the application of oscillating strains to a material allows to obtain two dynamic moduli, namely, the storage modulus, G′, a measure of the elasticity, and the loss modulus, G″, representing viscous components, at a given frequency, w, of oscillation. A traditional gel will typically exhibit a semi-solid-like or solid-like mechanical spectrum, that is, G′>G″ throughout the linear viscoelastic region.

In various embodiments, gel compositions according to the disclosure may initially have the appearance of a traditional gel, and be semi-clear or substantially clear. The viscosity of the gel is not limited, but by way of example only, in certain embodiments, the viscosity may range from about 1 to about 25 Pa*s, such as about 3.5 to about 23 Pa*s, about 5 to about 15 Pa*s, about 8 to about 10 Pa*s, or about 9 Pa*s, including all ranges and subranges thereof, at a shear rate of 2 1/s, when measured using a rheometer at about 25° C. with a 40 mm parallel plate and 1000 μm gap. In further embodiments, the viscosity of the gel may range from about 0.1 to about 25 Pa*s, such as about 0.1 to about 23 Pa*s, about 0.1 to about 15 Pa*s, or about 0.1 to about 10 Pa*s, including all ranges and subranges thereof, at a shear rate of 2 1/s, when measured using a Rheomat with an M2 spindle at about 25° C., for a time period of about 30 seconds.

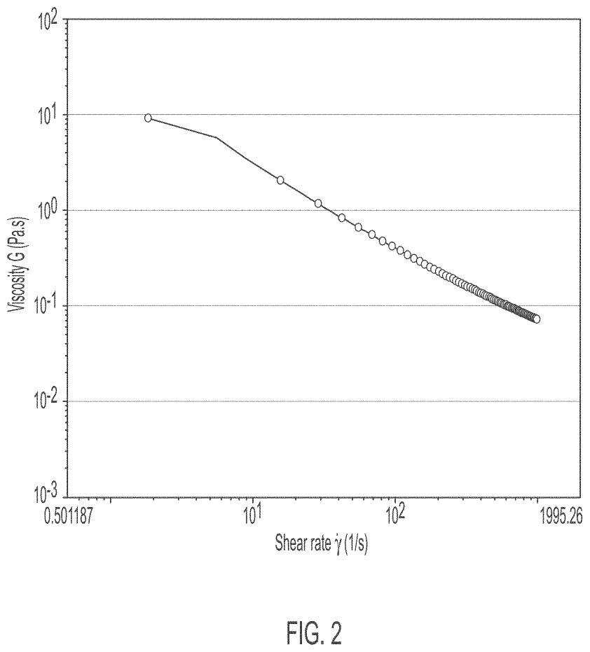

As seen in FIG. 2, gel compositions according to the disclosure typically have a viscosity of greater than about 10⁻¹ Pa*s at shear rates typically employed for hair compositions. However, it is understood by those skilled in the art that, at such viscosities, traditional gel compositions are difficult to transform into a foam in contrast to gel compositions according to the disclosure. By way of non-limiting example only, in one embodiment, the viscosity of the gel composition according to the disclosure may range up to about 10 Pa*s, and upon application of shear stress, the viscosity of the composition decreases to less than 0.1 Pa*s and a foam is formed.

In at least certain embodiments, the compositions in gel form may be stable, meaning that no or essentially no phase separation or significant change in pH or viscosity is seen, over a period of at least one week, such as at least one month, at least 6 months, or at least one year under ambient conditions.

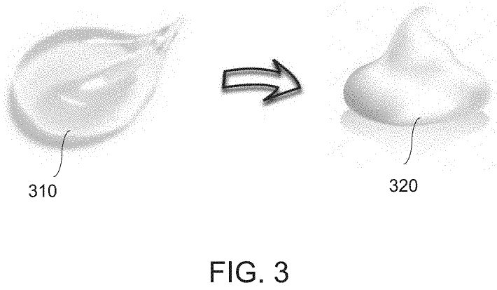

Compositions in the form of a gel according to the disclosure are able to transform into a foam (i.e. they have decreased viscosity) upon application of sufficient shear stress. For example, initially compositions according to the disclosure will exhibit a typical gel-like texture 310, as illustrated in FIG. 3, when prepared. Under application of shear stress, the compositions in the form of a gel 310 will transform to a foam 320. By way of example, mechanical shear stress, such as that which is applied by a pump dispenser, may effect the transformation of the gel to a foam. Other means of applying shear stress, for example shaking the composition, may also cause the gel to transform into a foam.

One way in which foam density is described herein relates to the volume of air bubbles to the total volume of the foam, which includes the bubbles and the liquid carrier thereof. The foam of compositions according to the disclosure may thus comprise air bubbles in at least about 30%, such as at least about 40%, of the foam, and the density may thus be described as at least about 30% or at least about 40% v/v. In various embodiments, the foams may be creamy and/or fine-pored. In certain embodiments, the foam may be sufficiently stable such that it remains substantially foam-like as it is rubbed between the hands and/or worked into the hair. In at least some embodiments, the foam of compositions according to the disclosure may remain stable for more than 5 minutes, such as more than 10 minutes, more than 20 minutes, more than 30 minutes, more than 45 minutes, more than 60 minutes, or more than 90 minutes, without a significant change in volume and/or density.

The foam compositions according to the present disclosure are generally easy to spread throughout the hair, which may improve coverage. The compositions further are able to provide style and/or shaping, and in at least in some embodiments may provide care benefits to hair, especially curly hair. The compositions may also have cosmetic attributes such as curl definition and elongation, as well as frizz control, shine, maintaining hydration of over 8 hours in some cases, ease of styling, no crunch, no build up, fast drying, and/or are not sticky.

II. Packaging Systems

The present disclosure also relates to packaging systems comprising the compositions described herein. The packaging systems comprise at least one container suitable for containing and/or applying the composition and/or transforming the compositions from a gel into a foam.

In various embodiments, a packaging system according to the present disclosure may include a dispensing container containing a composition disclosed herein. The composition contained in the dispensing container is generally transparent or semi-transparent, i.e., clear or semi-clear and in the form of a gel.

Compositions may optionally be in aerosol or non-aerosol form. If they are in aerosol form, the compositions or packaging systems may comprise at least one propellant. If they are in non-aerosol form they do not need a propellant. Instead, they can be packaged in any dispensing container that has a dispensing device configured to dispense its contents as non-aerosol foam, optionally as a controlled amount or dose.

By way of non-limiting example, suitable dispensing containers may be any type of non-aerosol foam dispensing container. Such container may include a foaming chamber having a mesh therein and/or a foam distributing nozzle, so called foam pump, pump dispenser, or pump foamer. The dispensing container may be any size or shape and may be made of any suitable material(s).

The dispensing container may be prefilled with a gel composition according to the disclosure. The dispensing container may also be configured to allow filling or refilling with a composition disclosed herein when the dispensing container is empty or when the composition contained therein is running low, by the user at any time or near the time of use.

III. Methods

The present disclosure also relates to methods for styling keratin fibers, e.g. human hair, using the compositions described herein. The methods generally comprise applying any of the disclosed compositions to the keratin fibers. According to the present disclosure, compositions described herein are particularly useful for styling, shaping, and/or defining curly hair.

In various exemplary embodiments, the methods may include applying a hair styling- or shaping-effective amount of a foam composition of the present disclosure to hair (wet, damp, or dry hair), before, during, and/or after styling and/or shaping the hair. The composition may be left on the hair for any period of time, such as a few hours or a few days, or until the next washing or rinsing of the hair.

In some embodiments, a composition of the disclosure may be applied to the hair and left on the hair, e.g. as a mousse for holding curls or other hair styles. In one embodiment, the hair is allowed to air dry after application of the composition, and is styled or shaped with no heat being applied to the hair. Styling or shaping the hair may involve the use of devices on hair such as a brush, a comb or running the fingers of the hand through the hair. Optionally, styling or shaping can be done using twisting out techniques. In this situation, the composition disclosed herein may be applied to the hair before or during performing twist out, where the hair is twisted and then let go to create curls or other hair styles.

In another embodiment, the methods may include caring for, styling, and/or shaping the hair with a heating device before, during, or after the composition is applied to the hair. However, in some embodiments, the methods of caring for, styling, and/or shaping the hair do not include or require the use of heat.

Additionally, the compositions provide a variety of desirable sensory benefits to the hair, for example, smoothness without weigh-down, detangling, frizz control, softness, moisture, manageability, and/or shine. As such, the compositions are useful in styling or shaping hair while also caring for, conditioning, and/or imparting one or more sensorial benefits to the hair. Accordingly, the instant disclosure encompasses methods for treating hair with the compositions of the instant disclosure. For example, the methods may also include applying the compositions to curly hair to boost volume or hold existing curl styles, as well as providing additional sensory benefits to the hair.

As described herein, due to the transformative texture or transformative nature of the gel compositions, the gel is transformed into a foam when a shear stress is applied to the gel. Thus, the disclosure also relate to methods of transforming gel compositions described herein into foams by applying shear stress to the gel.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” etc., are used interchangeably to denote that the listing of components immediately preceding the phrase, such as “A, B, C, D, or mixtures thereof” signifies that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin materials” and “applying a composition onto hair” and variations of these phrases are intended to mean contacting the keratin materials including hair and skin, with at least one of the compositions of the disclosure, in any manner. It may also mean contacting the keratin materials with an effective amount of the composition, e.g. an amount effective to style the hair, an amount effective to shape the hair, etc.

Unless otherwise indicated, all percentages herein are by weight, relative to the weight of the total composition.

As used herein, the term “conditioning” means imparting to hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, the term “styling” is intended to include “shaping.”

As used herein, the term “curly hair” refers to any hair including a curl. The curl may be natural or unnatural, i.e., formed by chemical treatment or physical treatment of the hair. The degree of curliness of the hair may vary and is not limited.

A “leave-in” composition or product refers to a composition such as a hair-treatment composition that is not rinsed and/or washed away with water or acceptable solvent after the application of the composition onto the keratin fiber, such as hair; instead, the composition is allowed to remain on the keratin fibers for a period of time as desired, such from 1 hour, 2 hours, 3 hours, 4 hours, up to 8 hours, overnight, or as long as needed, until next time of washing or rinsing the keratin fibers.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

As used herein, the term “transformative texture” or “transformative nature” refers to the texture or nature of compositions according to the disclosure that can be changed from a gel to a foam when a shear stress is applied to the gel.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1—Formulation of Gel Compositions

The following inventive gel compositions were prepared according to the formulations set forth in Table 1 below.

TABLE 1
Compositions

| | 1A | 1B | 1C | 1D | 1E | 1F | 1G | 1H |
|---|---|---|---|---|---|---|---|---|
| XANTHAN GUM | 1.0 | 1.0 | 1.0 | 1.0 | 1.0 | 1.0 | 1.0 | 1.0 |
| CAPRYLYL | 0.006 | 0.006 | 0.006 | 0.006 | 0.006 | 0.006 | 0.006 | 0.006 |
| GLYCOL | | | | | | | | |
| VP/DIMETHYL- | 0.2 | 0.2 | 0.2 | 0.2 | 0.2 | 0.2 | 0.2 | 0.2 |
| AMINOETHYL- | | | | | | | | |
| METH- | | | | | | | | |
| ACRYLATE | | | | | | | | |
| COPOLYMER | | | | | | | | |
| VP/METH- | 0.6 | 0.6 | 0.6 | 0.6 | 0.6 | 0.6 | 0.6 | 0.6 |
| ACRYL- | | | | | | | | |
| AMIDE/VINYL | | | | | | | | |
| IMIDAZOLE | | | | | | | | |
| COPOLYMER | | | | | | | | |
| HYDROXY- | 0.2 | 0.2 | 0.2 | 0.2 | 0.2 | 0.2 | 0.2 | 0.2 |
| PROPYL GUAR | | | | | | | | |
| GLYCERIN | 4.0 | 4.0 | 4.0 | 4.0 | 4.0 | 4.0 | 4.0 | 4.0 |
| CITRIC ACID | 0.1 | 0.1 | 0.1 | 0.1 | 0.1 | 0.1 | 0.1 | 0.1 |
| PEG-40 | 0.7 | 0.9 | 0.9 | 0.9 | 0.8 | 1.0 | 0.9 | 0.9 |
| HYDROGENATED | | | | | | | | |
| CASTOR OIL | | | | | | | | |
| SODIUM C14-16 | 1.14 | 1.14 | 1.14 | 1.14 | 1.14 | 1.14 | 1.14 | 1.14 |
| OLEFIN | | | | | | | | |
| SULFONATE | | | | | | | | |
| DECYL | 1.59 | 1.59 | 1.59 | 1.59 | 1.59 | 1.59 | 1.59 | 1.59 |
| GLUCOSIDE | | | | | | | | |
| VITAMINS | 0.1 | 0.1 | 0.1 | 0.1 | 0.1 | 0.1 | 0.1 | 0.1 |
| (panthenol, | | | | | | | | |
| tocopherol) | | | | | | | | |
| ADDITIVES | 1.96 | 1.85 | 1.05 | 1.96 | 1.96 | 1.96 | 1.16 | 1.96 |
| (preservatives, | | | | | | | | |
| pH adjusters, | | | | | | | | |
| fragrance, plant | | | | | | | | |
| extracts) | | | | | | | | |
| SOLVENT | QS to | QS to | QS to | QS to | QS to | QS to | QS to | QS to |
| (water + non- | 100 | 100 | 100 | 100 | 100 | 100 | 100 | 100 |
| aqueous | | | | | | | | |
| solvents) | | | | | | | | |

Compositions 1A-1H were prepared as follows. First, the water and hydroxypropyl guar were mixed in a homogenizer at low speed until the hydroxypropyl guar was well dispersed. The remaining components except the decyl glucoside and sodium C14-C16 olefin sulfonate were then added one at a time, mixing after each addition, until the mixture was uniform and the components were well dispersed. Finally, the decyl glucoside and sodium C14-C16 olefin sulfonate were added very slowly, and mixing continued until uniform.

Compositions 1A-1H were clear and looked like traditional hair gel compositions. The pH of each composition was in the range of 4.5 to 5.5. The viscosities of each of compositions 1A-1H were similar, and were in the range of 310-460 mPa*s, measured using a Rheomat with an M2 spindle for 30 seconds.

### Example 2—Evaluation of Beneficial Effects on Hair

A study was conducted to evaluate the performance of the composition of Example 1H in terms of properties imparted to the hair. The gel composition was first transformed to a stable, semi-transparent foam when a shear stress was applied. Once the foam was formed, the composition was applied to the hair of 54 panelists whose hair had been shampooed and conditioned, and was still damp when the composition was applied.

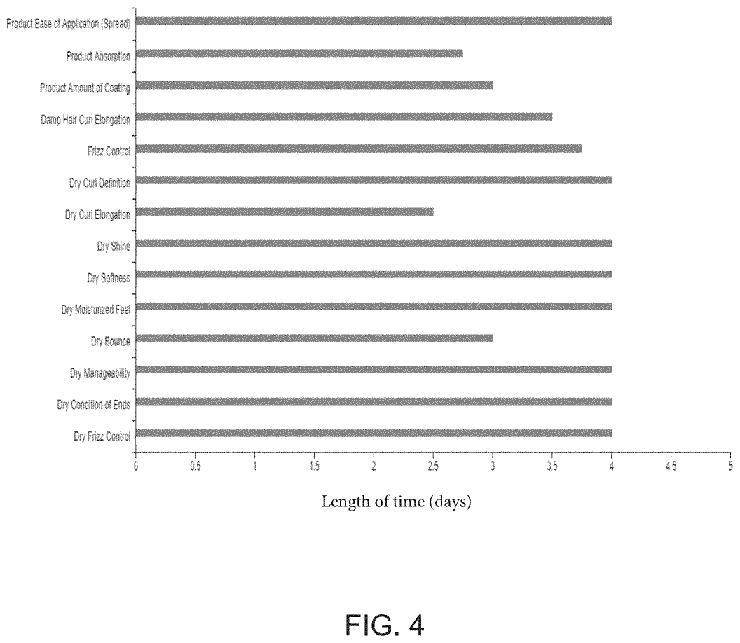

The panel gave the composition of Example 1H high ratings overall in terms of benefits such as curl definition, frizz control, shine, softness, ease of styling, manageability, and hold, while noting lack of tackiness of the composition and lack of greasiness, flaking, crunchiness, stiffness, or feeling weighed down of the hair, at TO (immediately upon application), T24H (24 hours after application), and T48H (48 hours after application). FIG. 4 is a graph showing how long the panelists reported various benefits lasted.

It will be apparent to those skilled in the art that various modifications and variations can be made in the compositions, methods, and packaging systems according to the disclosure without departing from the spirit or scope of the disclosure. Thus, it is intended that the disclosure cover such modifications and variations and their equivalents.

1. A gel composition comprising:
(a) at least one nonionic guar polymer;
(b) at least one polysaccharide other than the at least one nonionic guar polymer;
(c) at least one nonionic film-forming polymer;
(d) at least one cationic film-forming polymer;
(e) at least one anionic surfactant; and
(f) at least one polyol;
wherein the weight ratio of the at least one polysaccharide other than the nonionic guar polymer to the at least one nonionic guar polymer ranges from about 1:0.01 to about 1:1; and
wherein the gel composition has a transformative texture that can be transformed into a foam when shear stress is applied.

2. The composition according to claim 1, wherein the at least one nonionic guar polymer comprises a nonionic hydroxyalkyl-modified guar polymer.

3. The composition according to claim 1, wherein the at least one nonionic guar polymer is present in an amount ranging from about 0.01% to about 2% by weight, relative to the total weight of the composition.

4. The composition according to claim 1, wherein the at least one polysaccharide other than the at least one nonionic guar polymer is chosen from gums, celluloses, starches, or mixtures thereof.

5. The composition according to claim 1, wherein the at least one polysaccharide other than the at least one nonionic guar polymer is present in an amount ranging from about 0.01% to about 5% by weight, relative to the total weight of the composition.

6. The composition according to claim 1, wherein the at least one nonionic film-forming polymer is chosen from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, copolymers of acrylonitrile and of a non-ionic monomer, or a mixture thereof.

7. The composition according to claim 1, wherein the at least one nonionic film-forming polymer is present in an amount ranging from about 0.01% to about 5% by weight, relative to the total weight of the composition.

8. The composition according to claim 1, wherein the at least one cationic film-forming polymer is chosen from quaternized or non-quaternized vinylpyrroli-done/dialkylaminoalkyl acrylate or methacrylate copolymers, fatty-chain polymers containing a vinylpyrrolidone unit, dimethylaminoethyl methacrylate/vinylcaprolactam/vinylpyrrolidone terpolymers, quaternized vinylpyrrolidone/dimethylaminopropylmethacrylamide copolymers, cationic guar gum derivatives, quaternary copolymers of vinylpyrrolidone and of vinylimidazole, chitosans or salts thereof, cationic cellulose derivatives, or mixtures thereof.

9. The composition according to claim 1, wherein the at least one cationic film-forming polymer is present in an amount ranging from about 0.01% to about 5% by weight, relative to the total weight of the composition.

10. The composition according to claim 1, wherein the at least one anionic surfactant is chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof.

11. The composition according to claim 1, wherein the at least one anionic surfactant is present in an amount ranging from about 0.01% to about 6% by weight, relative to the total weight of the composition.

12. The composition according to claim 1, wherein the at least one polyol comprises at least one alkane polyol, present in an amount ranging from about 0.1% to about 15% by weight, relative to the total weight of the composition.

13. The composition according to claim 1, further comprising at least one alkypolyglucoside.

14. A gel composition according to claim 1, comprising:
(a) from about 0.01% to about 1% by weight of at least one nonionic hydroxyalkyl-modified guar polymer;
(b) from about 0.01% to about 3% by weight of at least one polysaccharide other than the at least one nonionic guar polymer chosen from gums, celluloses, starches, or mixtures thereof;
(c) from about 0.01% to about 2% by weight of at least one nonionic film-forming polymer;
(d) from about 0.1% to about 4% by weight of at least one cationic film-forming polymer;
(e) from about 0.01% to about 3% by weight of at least one anionic surfactant chosen from non-sulfate anionic surfactants comprising alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof; and
(f) from about 2% to about 6% by weight at least one polyol chosen from alkane polyols;
wherein the weight ratio of the at least one polysaccharide other than the nonionic guar polymer to the at least one nonionic guar polymer ranges from about 1:0.01 to about 1:1; and
wherein the gel composition has a transformative texture that can be transformed into a foam when shear stress is applied.

15. A packaging system comprising a dispensing container containing a gel composition comprising:
(a) at least one nonionic guar polymer;
(b) at least one polysaccharide other than the at least one nonionic guar polymer;
(c) at least one nonionic film-forming polymer;
(d) at least one cationic film-forming polymer;
(e) at least one anionic surfactant; and
(f) at least one polyol;
wherein the weight ratio of the at least one polysaccharide other than the nonionic guar polymer to the at least one nonionic guar polymer ranges from about 1:0.01 to about 1:1; and
wherein the gel composition has a transformative texture that can be transformed into a foam when shear stress is applied; and
wherein the dispensing container comprises a non-aerosol dispensing device configured to provide shear stress to the gel composition sufficient to transform the form of the gel composition into a foam.

16. A method for caring for, styling, and/or shaping hair, the method comprising:
(i) applying shear stress to a gel composition comprising:
(a) at least one nonionic guar polymer;
(b) at least one polysaccharide other than the at least one nonionic guar polymer;
(c) at least one nonionic film-forming polymer;
(d) at least one cationic film-forming polymer;
(e) at least one anionic surfactant; and
(f) at least one polyol;
wherein the weight ratio of the at least one polysaccharide other than the nonionic guar polymer to the at least one nonionic guar polymer ranges from about 1:0.01 to about 1:1; and
wherein the gel composition has a transformative texture that can be transformed into a foam when shear stress is applied; and
(ii) applying the foam to the hair.

17. The method according to claim 16, wherein the at least one nonionic guar polymer comprises a nonionic hydroxyalkyl-modified guar polymer, and is present in an amount ranging from about 0.01% to about 2% by weight, relative to the total weight of the composition.

18. The method according to claim 16, wherein the at least one polysaccharide other than the at least one nonionic guar polymer is chosen from gums, celluloses, starches, or mixtures thereof, and is present in an amount ranging from about 0.01% to about 5% by weight, relative to the total weight of the composition.

19. The method according to claim 16, wherein the at least one polyol comprises at least one alkane polyol, and is present in an amount ranging from about 0.1% to about 15% by weight, relative to the total weight of the composition.

20. The method according to claim 16, wherein the composition further comprises at least one alkypolyglucoside.
